(19) United States
(12) Patent Application Publication
(10) Pub. No.: US 2018/0042953 A1
(43) Pub. Date: Feb. 15, 2018

(54) ANTIBACTERIAL INJECTABLE PHARMACEUTICAL COMPOSITION

(71) Applicant: OBSHCHESTVO S OGRANICHENNOY OTVETSTVENNOSTYU "VIK - ZDOROVYE ZHIVOTNYKH", Moscow (RU)

(72) Inventors: Boris Viktorovich VIOLIN, Moscow (RU); Sergey Vyacheslavovich SEMENOV, Saratov (RU); Dmitriy Vladimirovich PRISTENSKIY, pos. Ust-Kurdyum (RU); Aleksandr Andreevich ANISKOV, Saratov (RU)

(21) Appl. No.: 15/792,970

(22) Filed: Oct. 25, 2017

Related U.S. Application Data
(63) Continuation-in-part of application No. 15/630,151, filed on Jun. 22, 2017.
Application No. 15/630,151 is a continuation of application No. 14/395,483, filed on Oct. 18, 2014, now abandoned.

(30) Foreign Application Priority Data
Jun. 8, 2012 (RU) 2012123616

(51) Int. Cl. A61K 31/7052; A61K 47/20; A61K 47/18; A61K 31/167; A61K 9/00; A61K 45/06; A61K 31/445; A61K 31/245; A61K 47/10; A61K 47/12

(52) U.S. Cl. CPC A61K 31/7052; A61K 47/10; A61K 47/20; A61K 47/18; A61K 47/12; A61K 9/0019; A61K 45/06; A61K 31/445; A61K 31/245; A61K 31/167; A61K 2300/00

(57) ABSTRACT

The invention relates to the field of veterinary science and medicine and can be used for the prophylaxis and treatment of bacterial infections. The aim of the present invention is to design a preparation based on azithromycin in the form of a solution, having a wide spectrum of antimicrobial activity, and in the form of an injection solution ready for use not only for introduction intravenously, but also for intramuscular, subcutaneous, intrauterine and intracisternal introduction. The antibacterial pharmaceutical composition comprises azithromycin, a pH adjuster, solvents and/or cosolvents, a preservative, and an antioxidant. The antioxidant is in the form of thioglycerol, sodium ascorbate, 4-methyl-2,6-di-tert-butylphenol, 2,4,5-trihydroxybutyrophenone, and combination thereof, whereas the preservative is in the form of chloroethon or benzyl alcohol with the following ratios of components by percentage mass: 5-30% azithromycin, up to 5% pH adjuster, 0.5-1% preservative, 0.1-0.2% antioxidant, the remainder being solvents.

### CROSS REFERENCE TO RELATED APPLICATIONS

The instant application is a Continuation-in-part application of U.S. patent application Ser. No. 15/630,151 filed Jun. 22, 2017, which is a continuation of Ser. No. 14/395,483 filed Oct. 18, 2014, which is a National Stage application of PCT application No. PCT/RU2013/000467 filed Jun. 6, 2013, which claims priority to Russian Patent Application No. 2012123616, filed Jun. 8, 2012, all of which incorporated herein by reference in their entireties. All said applications and their disclosures are incorporated herein by reference in their entireties.

### FIELD OF THE INVENTION

The invention relates to medicine and veterinary and can be used for the prevention and treatment of bacterial infections.

### BACKGROUND

In recent years, azithromycin with its high antimicrobial activity has been widely used in both medicine and veterinary medicine, an antibiotic from the group of macrolides.

Azithromycin, (2R,3S,4R,5R,8R,10R,11R,12S,13S,14R)-13-[(2,6-dioxy-3-C-methyl-3-O-methyl-a-L-ribohexopyranozyl)oxy]-2-ethyl-3, 4, 10-trihydroxy-3,5,6,8,10,12,14-heptamethyl-11-[[3,4,6-trideoxy-3-(dimethyl amino)-b-D-xylo-hexapyranozyl]oxy]-1-ox-6-azacyclopentadecane-15-one), is a semi-synthetic antibiotic, a derivative of erythromycin. Azithromycin is active against some gram-positive bacteria: Streptococcus spp. (Groups C, F, and G, except erythromycin-resistant ones), Streptococcus pneumoniae, Streptococcus pyogenes, Streptococcus agalactiae, Streptococcus viridans, Staphylococcus epidermidis, Staphylococcus aureus; against gram-negative bacteria: Haemophilusinfluenzae, Moraxella catarrhalis, Bordetella pertussis, Bordetella parapertussis, Legionella pneumophila, Haemophilusducreyi, Campylobacter jejuni, Neisseria gonorrhoeae and Gardnerella vaginalis; some anaerobes: Bacteroidesbivius, Clostridium perfringens, Peptostreptococcus spp; and Chlamydia trachomatis, Chlamydia pneumoniae, Mycoplasma pneumoniae, Mycobacterium avium complex, Ureaplasmaurealyticum, Treponema pallidum, Borreliaburgdorferi.

As is known, there exist various dosage forms (DF) of antibiotics, namely: for oral, parenteral and topical use. A special place among the developments of dosage forms is occupied by soluble dosage forms to ensure a uniform dispersion of particles. The dosage forms of antibiotics in the form of injectable solutions exhibit significantly increased absorption rates and degrees as compared to such drugs made as tablets, pastes, capsules or suspensions which must be disintegrated, dissolved and diffused within a limited period of time. Subcutaneous, intramuscular and intravenous administration of medicinal substances is carried out by injection, thus achieving the quickness of action upon the body and entering the blood in an unchanged form.

Safety (non-toxicity) and stability are basic requirements for injectable solutions. During the preparation and storage of solutions, many medicinal substances are destroyed to form inactive and toxic products under the influence of many factors. For example, in the process of sterilization of injection solutions and their subsequent storage, decomposition of the drug molecules is possible. Therefore, at present, the problem of the stability of dosage injectable forms in the conditions of expanding their range and increasing output is extremely urgent. Solving the problem of stability can be achieved by studying the nature of the reactions proceeding in certain dosage forms, by using physical and chemical methods of analysis for this purpose.

The preparation of stable nontoxic solutions means the maximum elimination of factors contributing to the decomposition of drugs, which is achieved by the use of auxiliary substances—stabilizers (antioxidants, preservatives, and pH regulators). Rational selection of the stabilizer will allow preparing a high-quality and therapeutically effective injectable dosage form.

Currently, azithromycin is available in few dosage forms for enteral (tablets, capsules, powder for suspension) and parenteral (lyophilizate for infusion solutions) use. Liquid azithromycin-based compositions for ophthalmic use are also known.

For example, a liquid pharmaceutical formulation with azithromycin is known for ophthalmic use with the active substance content up to 10% (U.S. Pat. No. 6,277,829 B 1 of Aug. 21, 2001; Antonio Asero, Grazia Mazzone, process for preparation of aqueous formulation for ophthalmic use). Another liquid azithromycin composition for topical application is also known (U.S. Pat. No. 7,064,104 B1 of Jun. 20, 2006 Jacques Luyckx, Frederic Pilotaz, Pharmaceutical composition based on macrolides for topical application in ophthalmology).

However, it should be noted that, with all the similarity of the processes of stabilization of injectable solutions and eye drops, there are differences due to the specific application of the latter, as well as by different manufacturing techniques. The isotonicity of ophthalmic solutions and the correspondence of the pH value of eye drops with that of the lacrimal fluid are necessary conditions in the preparation of eye drops. In U.S. Pat. No. 6,277,829, buffer solvents are used to adjust pH, but their effect on the stability of the dosage form was not investigated. Moreover, data on the stability of azithromycin in the dosage form of eye drops at a temperature of 60° C. for a month or longer are given, although it is known that for prolonged (longer than 3-6 months) storage, chemical reactions in a sample of the injectable dosage form as solution may give a large number of various unpredictable impurities, which will adversely affect both the components of the preparation and the overall toxicity of the dosage form.

A fast-dissolving form of azithromycin for oral use is known (U.S. Pat. No. 7,572,773 B2 of Aug. 11, 2009 Aleksandar Danilovsky, Nezevie Zdravka, single dose fast dissolving azithromycin).

Azithromycin salts with malonic acid have been isolated. These compounds can be used for making fast-dissolving forms of azithromycin for per oral applications (U.S. Patent Appl. No 2009/0318375 A1 of Dec. 24, 2009 Bo Sung Kwon, Eun Sook Kim, Hee Cheol Kim, Sangmin Yun, Myoung-sill Ko, Tae Hun Song, Han Kyong Kim, Kwee Hyun Suh, Gwansum Lee, crystalline azithromycin L-malate monohydrate and pharmaceutical composition containing same).

Various solid pharmaceutical compositions of azithromycin for oral use are known (U.S. Patent Appl. No 2007/0185194 A1 of Aug. 9, 2007 Kamal Mehta, Rajeev Shankar Mathur, Sujata Paul, Sanjeev Kumar Sethi, Rajiv Malik, stable oral compositions of azithromycin monohydrate; U.S. Patent Appl. No 2005/0106239 A1 of May 19, 2005 Ruth Tenengauzer, Joseph Schwarz, Julia Hrakovsky, Tania Lessen, Lev Khondo, Mathi Mathivanan, Claude Singer, Michal Pesachovich, stabilized azithromycin compositions; U.S. Patent No 2008/0096831 A1 of Apr. 24, 2008 Mohsen Sadatrezaei, Pablo Davila, Gary Barbera, stabilized azithromycin composition).

All the above compositions are intended for oral or topical use.

A solid dosage form comprising lyophilized azithromycin is known (U.S. Patent Appl. No 2006/0116336 of Jun. 1, 2006 Byung But Woo, K. Keith Knwok, Kang Yong Yang, lyophilized azithromycin formulation). There exists a lyophilized form of azithromycin under the commercial name “Surnamed”, Pliva production.

This dosage form is a sterile powder to prepare injection solution immediately prior to use because the antibiotic in solution rapidly decomposes.

The liquid injectable form of azithromycin, comprising ethyl alcohol, cosolvents (polyethanediol, propylene glycol), a pH regulator is the most similar to our proposed solution (see patent CN 1270723 C “Azithromycin injection and its preparation”).

However, this formulation contains ethanol; therefore it cannot be administered intramuscularly or subcutaneously because this would lead to tissue necrosis. It is only used intravenously and in dilute aqueous solution. Hence, it is not a ready-to-use injectable solution but a concentrate to be added to a solution for intravenous infusion.

A stable pasty composition is known for a wide variety of veterinary and pharmaceutical products (U.S. Pat. No. 6,787,342 of Sep. 7, 2004, Chen Jun, Paste formulations). This composition includes azithromycin, colloidal silicon dioxide, a viscosity modifier, and a carrier (solvent). Additionally, the composition may contain antioxidants and preservatives. The carrier is a liquid phase to dissolve the active substance. The solvents used include water, propylene glycol, and N-methylpyrrolidinone.

However, the solvent in this case serves to ensure homogeneity and bioavailability of the contents of the pasty composition, its effect on stability has not been studied. The viscosity modifiers in the composition, acting as cross-linking agents, ensure the interaction of the silica and the carrier to form an ointment-like/pasty product not ready for injection. To use this composition in the form of injections, it is dissolved before use, using water for injection or other sterile liquids as a solvent. And the authors do not investigate the stability and toxicity of the dosage form (azithromycin solution for injections), in particular, they do not measure the concentration of azithromycin at several shelf life, give no data on the acute toxicity of the dosage form at the beginning and end of its shelf life, which are necessary for the state registration of injectable solutions.

Multiple chemical reactions in the DF solution for a long storage period greatly complicate selection of the optimal chemically stable non-toxic DF composition by conventional methods of combining variants (combinations of the components). Therefore, studying of azithromycin solution stabilization is an important technological task.

### SUMMARY

The present invention is aimed at the development of an azithromycin preparation as a water-organic solution with a broad spectrum of antimicrobial activity, as a ready-to-use injection solution for intramuscular, subcutaneous, intrauterine and intracisternal administration as well as for intravenous administration.

The technical result is a high physicochemical stability (at least 18 months) and low toxicity of our liquid dosage form, what providing safety and efficiency of the treatment and prevention of diseases caused by pathogenic microorganisms.

An anhydrous solution of the azithromycin injectable preparation is known to have an increased toxicity and a strong local irritant effect. That is why a composition containing water and an organic solvent with minimal toxicity has been found, which is highly stable.

To reveal an effective and safe (non-toxic) combination of components, a comprehensive scientific study was made to identify chemical factors affecting the azithromycin solution stability and to determine the antioxidant/pH/organic solvent ratios for obtaining a stable and safe azithromycin dosage form.

The present invention provides a ready-for-use antibacterial injectable pharmaceutical composition, comprising:

azithromycin, present in a weight percentage in a range of 5-30%;

a pH adjuster, present in a weight percentage up to 5%;

a preservative, present in a weight percentage in a range of 0.5-1%, wherein the preservative is chloroethon or benzyl alcohol;

an antioxidant, present in a weight percentage in a range of 0.1-0.2%, wherein the antioxidant is selected from the group consisting of thioglycerol, sodium ascorbate, 4-methyl-2,6-di-tert-butylphenol, 2,4,5-trihydroxybutyrophenone, and combination thereof;

organic solvents present in a weight percentage in a range of 30-74%,

water present in a balance of the weight percentage;

wherein a concentration of the azithromycin present in concentration of at least 89% of the initial concentration of the azithromycin after 540 days of the composition shelf life.

More preferred are compositions comprising:

azithromycin, present in a weight percentage in a range of 10-20%;

a pH adjuster, present in a weight percentage in a range of 1.6 to 3.2%;

a preservative, present in a weight percentage in a range of 0.5-1.0%, wherein the preservative is chloroethon or benzyl alcohol;

an antioxidant, present in a weight percentage in a range of 0.1-0.2%, wherein the antioxidant is selected from the group consisting of thioglycerol, sodium ascorbate, 4-methyl-2,6-di-tert-butylphenol, 2,4,5-trihydroxybutyrophenone, and combination thereof;

organic solvents or co-solvents present in a balance of the weight percentage;

wherein a concentration of the azithromycin present in concentration of at least 89% of the initial concentration of the azithromycin after 540 days of the composition shelf life.

Also preferred are compositions comprising:

azithromycin, present in a weight percentage in a range of 10-20%;

a pH adjuster, present in a weight percentage in a range of 1.6 to 3.2%;

a preservative, present in a weight percentage in a range of 0.5%, wherein the preservative is chloroethon;

an antioxidant, present in a weight percentage in a range of 0.1%, wherein the antioxidant is selected from the group consisting of thioglycerol, sodium ascorbate, 4-methyl-2,6-di-tert-butylphenol, 2,4,5-trihydroxybutyrophenone, and combination thereof;

organic solvents or co-solvents present in a balance of the weight percentage;

wherein a concentration of the azithromycin present in concentration of at least 89% of the initial concentration of the azithromycin after 540 days of the composition shelf life.

In the compositions, the active substance is dissolved in a mixture of organic solvent and water with substances to stabilize the acidity of the solution added. The ratio of the organic solvent, water, and pH (acidity) regulators is selected in such a way that the physicochemical stability of the solution was as high as possible with the minimum degradation degree of the dissolved azithromycin. The organic solvent and pH adjuster are selected for the lowest toxicity when intramuscular or subcutaneous administration.

### BRIEF DESCRIPTION OF THE DRAWINGS

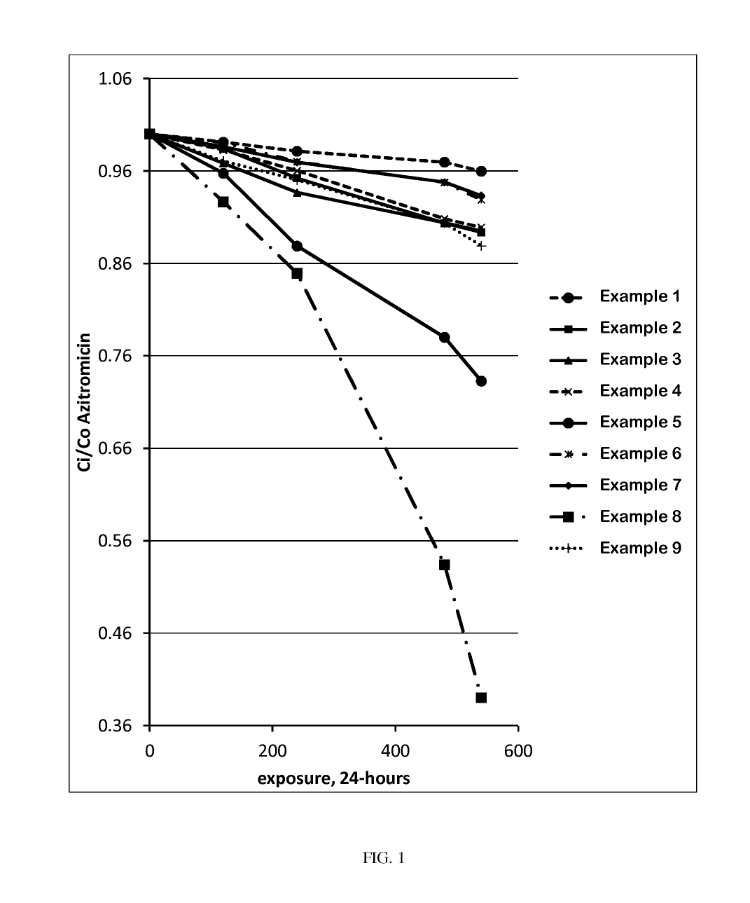

The invention is illustrated by a graph on FIG. 1 which shows data on the degradation of azithromycin solutions produced by the above formulas and examples based on the data from Table 7.

### DETAILED DESCRIPTION OF THE PREFERRED EMBODIMENTS

The development of our claimed dosage form was based on the following criteria:

1. Searching for the composition of the dosage form which provides the maximum possible concentration of azithromycin in solution; 2. Identification of substances (approved for use in injecting preparations) providing:
the maximum stability of the resulting solution (e.g. no precipitate, no solution delamination, etc.);
the maximum chemical stability of the active substance (azithromycin and other macrolides) in the proposed dosage form;

3. Selection of the dosage form with the minimum toxicity while retaining the high antibacterial activity inherent to azithromycin and other macrolides.

The developed formulation is stable solution of the active ingredient (azithromycin). Dimethylacetamide or dimethylsulfoxide or N-methyl-2-pyrrolidone or 2-pyrrolidone, or combinations thereof, or combinations of organic solvents with water are used as solvents or co-solvents. Citric acid or malic acid or benzoic acid or succinic acid, or combinations thereof are used as pH adjusters. Benzyl alcohol, parabens, monochlorethone, or combinations thereof are used as preservatives. Ascorbic acid or sodium ascorbate or calcium ascorbate or palmityl ascorbate or 4-methyl-2,6-di-tert-butylphenol or tert-butyl hydroquinone or 2,4,5-trihydroxybutyrophenone or sodium metabisulphite or thioglycerol or tocopherol acetate, or combinations thereof are used as antioxidants.

The dosage form may include a solubilizer. Cremophor EL or Cremophor ELP (polyoxyethylene-glycerol-triricinoleate) or Cremophor RH40 (polyoxyethylene-glycerol-trihydroxystearate) or Tween 80 (polyoxyethylenesorbitanmonooleate) or Solutol HS 15 (polyethylene glycol-660-12-hydroxystearate) or Poloxamer F68 or Poloxamer F127 (polyoxyethylene-polyoxypropylene-block-copolymer), or combinations thereof are used as solubilizers. The dosage form may also include an anesthetic—lidocaine hydrochloride (up to 0.5%).

### Methods for Assessing the Physicochemical Stability of Azithromycin Solutions

The content of azithromycin in solution was measured by HPLC using the method prescribed by RF State Pharmacopoeia XII, FS “Azithromycin”.

To justify our selection of the most optimal solvent, the solubility of azithromycin in various organic solvents was measured (Table 1).

TABLE 1
Azithromycin solubility in organic solvents.

| Solvent | Maximum azithromycin conc., % |
|---|---|
| Dimethylsulfoxide | 26.4 |
| 2-Pyrrolidone | 28.9 |
| N-methyl-2-pyrrolidone | 31.2 |
| N-ethyl-2-pyrrolidone | 25.8 |
| N-ethoxy-2-pyrrolidone | 23.3 |
| N-octyl-2-pyrrolidone | 21.5 |
| Dimethylacetamide | 32.1 |
| 3,3-dimethyl-2-pyrrolidone | 26.4 |
| Propylene glycol | 13.4 |
| Transcutol | 10.2 |

N-methyl-2-pyrrolidinone and dimethylacetamide are most optimal solvents.

However, it is known from Jouyban A, Fakhree M A, Shayanfar A. [1] that N-methyl-2-pyrrolidone and dimethylacetamide exhibit high toxicity when used in pure form. These solvents have dehydrating ability leading to dehydration and tissue necrosis at the injection site. Adding water to a formulation based on organic solvents is known to dramatically reduce these toxic effects [1].

Nevertheless, the addition of water to the composition on the basis of organic solvents did not solve the problem of the physicochemical stability of the dosage form (precipitation was observed).

To solve this problem, experiments were conducted on selection of counterions (pH adjusters) to azithromycin, which would provide the physicochemical stability of the dosage form of azithromycin in a water-organic solution. The following acids permitted for usage in injectable solutions were selected as pH (acidity) adjusters: citric acid, benzoic acid, lactic acid, and phosphoric acid.

Main experimental results are shown in Table 2.

TABLE 2
Effect of counterions (pH adjusters) on the physicochemical stability of water-organic composition of azithromycin.

| Component | DF options, concentration, wt. % | | | | | | | |
|---|---|---|---|---|---|---|---|---|
| | I | II | III | IV | V | VI | VII | VIII |
| Azithromycin | 10 | 10 | 10 | 10 | 10 | 10 | 10 | 10 |
| Dimethylacetamide | 50 | 50 | — | — | 50 | 50 | — | — |
| N-methyl-2-pyrrolidon | — | — | 50 | 50 | — | — | 50 | 50 |
| Citric acid | 3-5 | — | 3-5 | — | — | — | — | — |
| Benzoic acid | — | 3-5 | — | 3-5 | — | — | — | — |
| Phosphoric acid | — | — | — | — | 3-5 | — | 3-5 | — |
| Lactic acid | — | — | — | — | — | 3-5 | — | 3-5 |
| Water for injections | to 100 | to 100 | to 100 | to 100 | to 100 | to 100 | to 100 | to 100 |
| Incubation period, days | Parameters of physicochemical stability | | | | | | | |
| 20 | Transparent | Transparent | Transparent | Transparent | Transparent | Transparent | Transparent | Transparent |
| | Colorless | Colorless | Colorless | Colorless | Colorless | Colorless | Colorless | Colorless |
| | solution | solution | solution | solution | solution | solution | solution | solution |
| 40 | Transparent | Transparent | Transparent | Transparent | Precipitate | Precipitate | Precipitate | Precipitate |
| | Colorless | Colorless | Colorless | Colorless | | | | |
| | solution | solution | solution | solution | | | | |
| 60 | Transparent | Transparent | Transparent | Transparent | Precipitate | Precipitate | Precipitate | Precipitate |
| | Colorless | light-yellow | light-yellow | Colorless | | | | |
| | solution | solution | solution | solution | | | | |

On the basis of the data obtained, the variants of dosage forms I and IV were selected as most physicochemically stable and causing no toxic effects when injected in experiments on laboratory animals. Further studies of these formulations were performed at longer incubation times with evaluation of the concentration of the active ingredient (azithromycin) (Table 3).

TABLE 3
Physicochemical stability of dosage forms I and IV at incubation up to 300 days at room temperature

| | Stability | Incubation, days | | | | | |
|---|---|---|---|---|---|---|---|
| No | parameters | 0 | 60 | 120 | 180 | 240 | 300 |
| | Precipitate | no | no | no | no | no | no |
| | Color, | Colorless | Colorless | Colorless | Light | Light | Light |
| | transparency | transparent | transparent | transparent | yellow | yellow | yellow |
| | of solution | | | | transparent | transparent | transparent |
| | Azithromycin] | 9.9 | 9.8 | 9.8 | 9.5 | 9.0 | 8.7 |
| | concentration, % | | | | | | |
| V | Precipitate | no | no | no | no | no | no |
| | Color, | Light | Light | Light | Light | Yellow | Yellow |
| | transparency | yellow | yellow | yellow | yellow | transparent | transparent |
| | of solution | transparent | transparent | transparent | transparent | | |
| | Azithromycin] | 10.3 | 10.3 | 10.1 | 9.3 | 9.3 | 8.9 |
| | concentration, % | | | | | | |

Azithromycin concentration was determined by the HPLC technique published in the State Pharmacopoeia XII [2] and the European Pharmacopoeia [3], with some modifications. It can be seen from Table 3 that the chemical stability of the dosage form chosen is insufficient.

At a next stage of our study the list of pH adjusters was expanded and experiments were conducted with ascorbic acid, on the basis of the same dosage forms. Most relevant results are presented in Table 4.

TABLE 4
Effect of ascorbic acid as a pH adjuster on the physicochemical stability of the
selected dosage forms.

| | Ascorbic | Incubation | Stability parameters | | |
|---|---|---|---|---|---|
| | acid | days at | Transparency, | | Azithromycin |
| No | concentration, % | t = 20° C. | color | Precipitate | concentration, % |
| I | 1 | 0 | Colorless | No | 10.2 |
| | | | transparent | | |
| | | 20 | Colorless | No | 9.8 |
| | | | transparent | | |
| | | 40 | Colorless | No | 9.6 |
| | | | transparent | | |
| | | 60 | Colorless weakly | No | 9.0 |
| | | | opalescent | | |
| I | 5 | 0 | Colorless | No | 9.9 |
| | | | transparent | | |
| | | 20 | Colorless weakly | No | 9.9 |
| | | | opalescent | | |
| | | 40 | Turbid white | No | 9.9 |
| | | 60 | Colorless | White | N/A |
| | | | | precipitate | |
| IV | 1 | 0 | Colorless | No | 10.0 |
| | | | transparent | | |
| | | 20 | Colorless weakly | No | 9.9 |
| | | | opalescent | | |
| | | 40 | Colorless weakly | No | 9.7 |
| | | | opalescent | | |
| | | 60 | Colorless weakly | No | 9.3 |
| | | | opalescent | | |
| IV | 5 | 0 | Colorless | No | 10.1 |
| | | | transparent | | |
| | | 20 | Colorless weakly | No | 10.1 |
| | | | turbid | | |
| | | 40 | Colorless | Small white | 10.0 |
| | | | | Precipitate | |
| | | 60 | Colorless | Precipitate | N/A |

These experiments show that ascorbic acid in high concentrations is effective as an azithromycin degradation inhibitor but provides no solution stability of the dosage form.

Having analyzed the whole set of the data obtained, it was decided to leave citric and benzoic acids as pH adjusters with introduction of small amounts of ascorbic acid as an additional counterion. The main results of this experiment are presented in Table 5. Dosage form I is taken for illustration (dimethylacetamide+citric acid).

TABLE 5
Effect of ascorbic acid on the stability of the dosage form

| | | Stability parameters | | |
|---|---|---|---|---|
| Ascorbic acid | Incubation, days, | Transparency, | | Azithromycin |
| concentration, % | at t 20° C. | color | Precipitate | concentration, % |
| 0.8 | 0 | Colorless | No | 9.9 |
| | | transparent | | |
| | 60 | Colorless turbid | No | 9.8 |
| | 120 | Colorless | White | N/A |
| | 180 | Colorless | White | N/A |
| 0.4 | 0 | Colorless | No | 9.8 |
| | | transparent | | |
| | 60 | Colorless | No | 9.8 |
| | | transparent | | |
| | 120 | Colorless | White | H/O |
| | 180 | Colorless | White | H/O |
| 0.2 | 0 | Colorless | No | 10.1 |
| | | transparent | | |
| | 60 | Colorless | No | 10.1 |
| | | transparent | | |
| | 120 | Colorless | No | 10.0 |
| | | transparent | | |
| | 180 | Colorless | No | 10.1 |
| | | transparent | | |
| 0.1 | 0 | Colorless | No | 9.8 |
| | | transparent | | |
| | 60 | Colorless | No | 9.8 |
| | | transparent | | |
| | 120 | Colorless | No | 9.8 |
| | | transparent | | |
| | 180 | Colorless | No | 9.8 |
| | | transparent | | |

Therefore, it the ratio of the pH regulator and ascorbic acid as an additional counterion was determined, providing a secure and stable dosage form of azithromycin for injection.

Ascorbic acid is known to be used as antioxidant in the pharmaceutical and food industry. In this connection, experiments were conducted with other antioxidants to reveal an effect similar to the action of ascorbic acid in the dosage forms selected. The data obtained are presented in Table 6.

TABLE 6
Examples of the effect of antioxidants on the stability of
dosage form I at the concentration of antioxidants 0.1-0.2%

| | | Stability parameters | | |
|---|---|---|---|---|
| | Incubation, | | | Azithromycin |
| | days at t | Transparency, | Precip- | concen- |
| Antioxidant | 20° C. | color | itate | tration, % |
| Sodium | 0 | Colorless | No | 10.4 |
| ascorbate | | transparent | | |
| | 20 | Colorless | No | 10.4 |
| | | transparent | | |
| | 40 | Colorless | No | 10.4 |
| | | transparent | | |
| Palmityl | 0 | Light yellow | No | 10.2 |
| ascorbate | | opalescent | | |
| | 20 | Light yellow | No | 10.2 |
| | | opalescent | | |
| | 40 | Light-yellow, | No | 10.2 |
| | | badly | | |
| | | opalescent | | |
| 4-methyl- | 0 | Colorless | No | 10.0 |
| 2.6-di-tert- | | transparent | | |
| butylphenol | 20 | Colorless | No | 10.0 |
| | | opalescent | | |
| | 40 | Colorless | No | 10.0 |
| | | opalescent | | |
| | 0 | Colorless | No | 9.9 |
| | | transparent | | |
| Sodium | 20 | Colorless | No | 9.9 |
| metabisulfite | | transparent | | |
| | 40 | Colorless | No | 9.9 |
| | | transparent | | |
| Thioglycerol | 0 | Colorless | No | 10.0 |
| | | transparent | | |
| | 20 | Colorless | No | 10.0 |
| | | transparent | | |
| | 40 | Colorless | No | 10.0 |
| | | transparent | | |

It is seen from Table 6 that the antioxidants tested can also act as an additional complexing counterion, simultaneously providing protection against oxidation.

The formation of a complex of the antibiotic with ascorbic acid or other similar substances is, in this case, the cause of stabilizing azithromycin and other macrolide antibiotics. The stability constant of the antibiotic/antioxidant complex is high enough, so such a complex exists a little time in an water-organic medium and does not have time to withdraw from the reaction (to precipitate). On the other hand, organic acids ionize the free molecule of azithromycin, and it again reacts with an additional counterion, resulting in equilibrium inherent in the solution of the formulation. Further development was to evaluate the range of the organic solvent/pH adjuster/additional complexing counterion (antioxidant)/active substance weight ratios and to optimize possible compositions of the dosage forms.

Method of Preparation

Azithromycin is suspended in an appropriate solvent or a solvent mixture, and then a pH adjustor, a solubilizer, an anesthetic, a preservative, and an antioxidant are added.

Below are several compositions of our dosage forms to illustrate the optimal ratio of the components:

### Example 1

A composition with an anesthetic is provided.

| Component | Amount, wt. % |
|---|---|
| Azithromycin | 10.0 |
| Lidocaine hydrochloride | 0.1 |
| N-methyl-2-pyrrolidone | 50.0 |
| Citric acid | 3.2 |
| Benzylalcohol | 1.0 |
| Methyl-2,6-di-tert-butylphenol | 0.1 |
| Water | Up to 100 |

### Example 2

A formulation is provided with a solubilizer (polyoxyethylene-glycerol-triricinoleate) and the corresponding amounts of other components.

| Component | Amount, wt. % |
|---|---|
| Azithromycin | 10.0 |
| Dimethylacetamide | 30.0 |
| Citric acid | 3.2 |
| Polyoxyethylene-glycerol- | 15.0 |
| triricinoleate | |
| Benzylalcohol | 1.0 |
| Sodium metabisulfite | 0.1 |
| Water | Up to 100 |

### Example 3

A formulation is provided with the maximum amount of the solubilizer and appropriate amounts of other components.

| Component | Amount, wt. % |
|---|---|
| Azithromycin | 10.0 |
| Dimethylacetamide | 35.0 |
| Citric acid | 3.0 |
| Polyoxyethylene-glycerol- | 30.0 |
| triricinoleate | |
| Benzylalcohol | 1.0 |
| Methyl-2,6-di-tert-butylphenol | 0.1 |
| Water | Up to 100 |

Increasing the amount of the solubilizing agent above 30% leads to instability of the solution (delamination and precipitation).

### Example 4

A formulation with the maximum amount of the organic solvent and appropriate amounts of other components is provided.

| Component | Amount, wt. % |
|---|---|
| Azithromycin | 20.0 |
| Dimethylacetamide | 74.0 |
| Citric acid | 1.6 |
| Benzylalcohol | 1.0 |
| Methyl-2,6-ditertbutylphenol | 0.1 |
| Water | Up to 100 |

An increased amount of the organic solvent (above 80%) leads to enhanced toxic properties of the dosage form.

### Example 5

A formulation with the maximum amount of organic solvent and appropriate amounts of other components, without pH control, is provided.

| Component | Amount, wt. % |
|---|---|
| Azithromycin | 20.0 |
| Dimethylacetamide | 74.0 |
| Benzylalcohol | 1.0 |
| Methyl-2,6-di-tert-butylphenol | 0.1 |
| Water | Up to 100 |

The elimination of the regulator from the formulation affects the stability of the solution (rapid darkening, a drop of the active ingredient concentration); in addition, an increased amount of the organic solvent (above 70%) leads to increased toxic properties of the dosage form.

### Example 6

A formulation is provided with poloxamer F127 as a solubilizer, with no addition of an organic cosolvent, and with appropriate amounts of other components.

| Component | Amount, wt. % |
|---|---|
| Azithromycin | 5.0 |
| Citric acid | 3.2 |
| PoloxamerF127 | 5.0 |
| Benzylalcohol | 1.0 |
| Sodium meta bisulfite | 0.1 |
| Water | Up to 100 |

A reduced amount of the solubilizer (below 5.0%) entails a decreased stability of the dosage forms (precipitation); besides, a reduced amount of the active agent leads to a decreased therapeutic efficacy of the formulation.

### Example 7

A formulation is provided with Poloxamer F127 as a solubilizer, with no addition of an organic cosolvent, and with more azithromycin than in example 6:

| Component | Amount, wt. % |
|---|---|
| Azithromycin | 10.0 |
| Citric acid | 3.2 |
| Poloxamer F127 | 8.0 |
| Benzyl alcohol | 1.0 |
| Sodium metabisulfite | 0.1 |
| Water | Up to 100 |

An increased azithromycin content (above 10.0%), when Poloxamer F127 is used as a solubilizer, leads to an increased viscosity of the solution at temperatures below +15° C.

### Example 8

A formulation is provided with the maximum amount of a pH regulator and appropriate amounts of other components.

| Component | Amount, wt. % |
|---|---|
| Azithromycin | 10.0 |
| Dimethylacetamide | 35.0 |
| Citric acid | 5.0 |
| Benzylalcohol | 1.0 |
| Ascorbic acid | 0.1 |
| Water | Up to 100 |

An increased amount of a pH regulator (above 5%) results in an increased acidity of the solution, and enhanced the local irritating effect of the dosage form enhances, and the degradation rate of the main component rises.

### Example 9

A formulation is provided with the maximum amount of the active substance and appropriate amounts of other components.

| Component | Amount, wt. % |
|---|---|
| Azithromycin | 50.0 |
| Dimethylacetamide | 43.9 |
| Citric acid | 5.0 |
| Benzylalcohol | 1.0 |
| Ascorbic acid | 0.1 |
| Water | Up to 100 |

When the concentration of azithromycin becomes more than 50%, the solution becomes insufficiently stable, and solutions with the concentration of the active substance within 30-50% are viscous enough, which deteriorates their operating properties (performance).

### Example 10

A formulation is provided with dimethylsulfoxide as an organic solvent.

| Component | Amount, wt. % |
|---|---|
| Azithromycin | 10.0 |
| Dimethylsulfoxide | 70.0 |
| Benzoic acid | 3.2 |
| Benzylalcohol | 1.0 |
| Ascorbic acid | 0.1 |
| Water | Up to 100 |

The forms with dimethylsulfoxide as the main solvent are less stable (precipitation).

### Example 11

A formulation is provided with 2-pyrrolidone as an organic solvent.

| Component | Amount, wt. % |
|---|---|
| Azithromycin | 10.0 |
| 2-pyrrolidone | 60.0 |
| Benzoic acid | 3.2 |
| Benzylalcohol | 1.0 |
| Ascorbic acid | 0.1 |
| Water | Up to 100 |

### Example 12

A formulation is provided where dimethylacetamide and benzyl alcohol are used as organic solvents.

| Component | Amount, wt. % |
|---|---|
| Azithromycin | 10.0 |
| Dimethylacetamide | 30.0 |
| Benzoic acid | 3.2 |
| Benzylalcohol | 20.0 |
| Ascorbic acid | 0.1 |
| Water | Up to 100 |

The forms with benzyl alcohol (or other alcohols) as a cosolvent are characterized by lower stability (color changes of the solution and degradation of the active ingredient).

### Example 13

A formulation is provided where chloroethon is used as preservative.

| Component | Amount, wt. % |
|---|---|
| Azithromycin | 10.0 |
| Lidocaine hydrochloride | 0.1 |
| N-methyl-2-pyrrolidone | 50.0 |
| Citric acid | 3.2 |
| Chloroethon | 0.5 |
| Methyl-2,6-di-tert-butylphenol | 0.1 |
| Water | Up to 100 |

A stability study was carried out for samples stored at ambient temperature (17-26° C.). The results are shown in Table 7.

TABLE 7

| | Exposure, days | | | | |
|---|---|---|---|---|---|
| | 0 | 120 | 240 | 480 | 540 |
| Example No | Azithromycin concentration, % | | | | |
| 1 | 10.17 | 10.08 | 9.98 | 9.86 | 9.76 |
| 2 | 10.05 | 9.89 | 9.57 | 9.08 | 8.98 |
| 3 | 10.10 | 9.78 | 9.46 | 9.13 | 9.04 |
| 4 | 20.68 | 20.32 | 19.86 | 18.78 | 18.59 |
| 5 | 20.46 | 19.59 | 17.98 | 15.96 | 14.99 |
| 6 | 5.34 | 5.29 | 5.18 | 5.06 | 4.96 |
| 7 | 10.17 | 10.03 | 9.86 | 9.64 | 9.49 |
| 8 | 10.22 | 9.47 | 8.68 | 5.46 | 3.99 |
| 9 | 50.54 | 49.06 | 48.03 | 45.67 | 44.42 |

From these data it follows that the dosage forms made according to the above formulations satisfy the requirements of physicochemical stability, Formulations 1-4 being the most optimal. Adding an anesthetic to the composition does not alter the physicochemical properties of the dosage form.

After 540 days of the composition shelf life the concentration of the azithromycin present in concentration of at least 89% of the initial concentration of the azithromycin.

From the graph it follows that addition of inadequate amounts of a pH adjuster leads to instability of the solution (a drop of the active ingredient concentration, Example 5) as well as the use of its excessive amounts (Example 8). Ignoring an organic solvent in the composition of the injectable solution with simultaneous addition of an appropriate solubilizer (poloxamer F 127), renders almost no effect on the solution stability (Examples 6, 7), but raises the solution viscosity noticeably. Inclusion of a solubilizer into the solution, with the simultaneous usage of an organic solvent, has no effect on the stability (Examples 1-4), but the presence of a surfactant may improve the pharmacological properties of the resulting formulations. An increased azithromycin content (up to 50%) (Example 9) causes the usage of an organic solvent with a pH adjuster only, and the use of surfactants is excluded due to the high viscosity of the forms with a solubilizer in their composition.

In addition to stability studies, the LD50 (acute toxicity) of the injectable dosage forms was tested at the end of their shelf life (Table 8). The initial LD50 (mouse, intraperitoneally) of DF Nos 1, 4 and 5 was 220-230 mg/kg of body weight (for azithromycin).

TABLE 8
Stability (content) of azithromycin and the acute toxicity
values at the end of exposure (540 days) in injectable
dosage forms. Incubation temperature + 17-6° C.

| | | | | | | Acute toxicity |
|---|---|---|---|---|---|---|
| | | | | | | LD50 (mice), mg/kg |
| | | | | | | of body weight for |
| | Exposure, days | | | | | azithromycin (intra- |
| | 0 | 120 | 240 | 480 | 540 | peritoneal injection, |
| Example No | Azithromycin concentration, % | | | | | storage 540 days) |
| 1 | 10.17 | 10.08 | 9.98 | 9.86 | 9.76 | 230 ± 23.1 |
| 4 | 20.68 | 20.32 | 19.86 | 18.78 | 18.59 | 192 ± 20.0 |
| 5 | 20.46 | 19.59 | 17.98 | 15.96 | 14.99 | 188 ± 19.1 |

Thus, the toxicity of the water-organic dosage forms presented in our application changes by the end of the shelf life by no more than 20%, which poses no danger with the great “therapeutic latitude” of azithromycin. It should be noted that toxicity cannot be predicted on the basis of any available data published in the open press, due to the complexity of bioactive molecules and the multiplicity of chemical reactions that can occur in the multicomponent solution for a long time, which indicates the nonobviousness of the proposed invention.

The compositions of the dosage forms presented in our application are 2 times less toxic than the anhydrous dosage form of azithromycin.

The new technical result, namely, the stability and low toxicity of our ready-for-use antibacterial injectable pharmaceutical composition in this application is achieved by identifying and optimizing new combinations of antioxidants, organic solvents, pH adjusters, and macrolide antibiotic (azithromycin). The combinations found provide obtaining injectable antibiotic solution that meets the essential requirements of pharmacopoeias, namely:
Physicochemical stability of both the dosage form (DF) and the active substance;
Low toxicity (safety);
Preservation of the declared (proper) antibacterial activity of the antibiotic.

It will be understood that the current invention may be embodied in other specific forms without departing from the spirit or central characteristics thereof. The present examples and embodiments, therefore, are to be considered in all respects as illustrative and not restrictive, and the invention is not to be limited to the details given herein.

What we claim are:

1. A ready-for-use antibacterial injectable pharmaceutical composition, comprising:
an azithromycin, present in a weight percentage in a range of 5-30%;
a pH adjuster, present in a weight percentage up to 5%;
a preservative, present in a weight percentage in a range of 0.5-1%, wherein the preservative is chloroethon or benzyl alcohol;
an antioxidant, present in a weight percentage in a range of 0.1-0.2%, wherein the antioxidant is selected from the group consisting of thioglycerol, sodium ascorbate, 4-methyl-2,6-di-tert-butylphenol, 2,4,5-trihydroxybutyrophenone, and combination thereof;
organic solvents present in a weight percentage in a range of 30-74%,
water present in a balance of the weight percentage;
wherein a concentration of the azithromycin present in concentration of at least 89% of the initial concentration of the azithromycin after 540 days of the composition shelf life.

2. The ready-for-use composition according to claim 1, wherein the organic solvents or co-solvents are selected from the group consisting of dimethylacetamide, 2-pyrrolidone, and combinations thereof.

3. The ready-for-use composition according to claim 1, further comprising a solublizer, wherein the solubilizer is selected from the group consisting of poloxamerF127, polyoxyethylene-glycerol-triricinoleate, and combination thereof, wherein the solubilizer is present in a weight percentage in the range of 1-30%.

4. The ready-for-use composition according to claim 1, further comprising an anesthetic, wherein the anesthetic present in a weight percentage up to 2.0%.

5. The ready-for-use composition according to claim 4, wherein the anesthetic is selected from the group consisting of sevicaine, bupivacaine, ropivacaine, lidocaine, and combination thereof.

6. The ready-for-use composition according to claim 1, wherein the composition is operable to be administered by intramuscular, subcutaneous, intrauterine, intracisternal or intravenous administration.

7. A ready-for-use antibacterial injectable pharmaceutical composition, comprising:
an azithromycin, present in a weight percentage in a range of 10-20%;
a pH adjuster, present in a weight percentage in a range of 1.6 to 3.2%;
a preservative, present in a weight percentage in a range of 0.5-1.0%, wherein the preservative is chloroethon or benzyl alcohol;
an antioxidant, present in a weight percentage in a range of 0.1-0.2%, wherein the antioxidant is selected from the group consisting of thioglycerol, sodium ascorbate, 4-methyl-2,6-di-tert-butylphenol, 2,4,5-trihydroxybutyrophenone, and combination thereof;
organic solvents or co-solvents present in a balance of the weight percentage;
wherein a concentration of the azithromycin present in concentration of at least 89% of the initial concentration of the azithromycin after 540 days of the composition shelf life.

8. A ready-for-use antibacterial injectable pharmaceutical composition, comprising:
an azithromycin, present in a weight percentage in a range of 10-20%;
a pH adjuster, present in a weight percentage in a range of 1.6 to 3.2%;
a preservative, present in a weight percentage in a range of 0.5%, wherein the preservative is chloroethon;
an antioxidant, present in a weight percentage in a range of 0.1%, wherein the antioxidant is selected from the group consisting of thioglycerol, sodium ascorbate, 4-methyl-2,6-di-tert-butylphenol, 2,4,5-trihydroxybutyrophenone, and combination thereof;
organic solvents or co-solvents present in a balance of the weight percentage;
wherein a concentration of the azithromycin present in concentration of at least 89% of the initial concentration of the azithromycin after 540 days of the composition shelf life.
